(19) United States
(12) Patent Application Publication
(10) Pub. No.: US 2024/0390197 A1
(43) Pub. Date: Nov. 28, 2024

(54) BODY-WEARABLE MEDICAL DEVICE COMPRISING A SUPERABSORBENT SUBSTANCE

(71) Applicant: Roche Diabetes Care, Inc., Indianapolis, IN (US)

(72) Inventors: Olaf Roscher, Schriesheim (DE); Harald von Campenhausen, Ladenburg (DE)

(21) Appl. No.: 18/798,243

(22) Filed: Aug. 8, 2024

Related U.S. Application Data
(63) Continuation of application No. PCT/EP2023/052913, filed on Feb. 7, 2023.

(30) Foreign Application Priority Data
Feb. 10, 2022 (EP) 22 156 206.9

(51) Int. Cl. A61F 13/58; A61F 13/53; A61F 13/531; A61M 5/172

(52) U.S. Cl. CPC A61F 13/58; A61F 13/531; A61M 5/1723; A61F 2013/530481; A61F 2013/530868

(57) ABSTRACT

A body-wearable medical device has a medical device housing having a transcutaneous element configured for being at least partially inserted into a patient's body at an insertion site during use of the body-wearable medical device. A base is configured for reversibly attaching the body-wearable medical device during its use to a patient's skin. The base has a first side that faces the patient's body and that also has an adhesive for adhering the body-wearable medical device to a patient's skin. For reducing a likelihood of contamination by body fluid flowing out of the insertion lesion, the base on its first side also has a superabsorbent substance and at least one fluid channel. The fluid channel is configured to guide body fluid from the insertion site to the superabsorbent substance.

### RELATED APPLICATIONS

This application is a continuation of International Application Serial No. PCT/EP2023/052913, filed Feb. 7, 2023, which claims priority to European Patent Application Serial No. 22 156 206.9, filed Feb. 10, 2022, the entire disclosures of both of which are hereby incorporated herein by reference.

### BACKGROUND

This disclosure relates to a body-wearable medical device having a medical device housing with a transcutaneous element that is configured for being at least partially inserted into a patient's body at an insertion site during use of the body-wearable medical device, and a base portion that is configured for reversibly attaching the body-wearable medical device during its use to a patient's skin, wherein the base portion has a first side that during use of the body-wearable medical device faces the patient's body and that also has an adhesive for adhering the body-wearable medical device to a patient's skin.

During use, a hitherto known body-wearable medical device is configured to be releasably attached to a patient's skin at an insertion site by means of the adhesive, wherein the transcutaneous elements is at least partially inserted into a patient's body. Upon inserting the transcutaneous element at least partially into the patient's body or during use of the body-wearable medical device, respectively, a body fluid such as, for example, blood, wound exudate or interstitial fluid may flow out of the insertion lesion that has been caused by the transcutaneous element. The flow of body fluids may contaminate not only the patient's clothes, but may also contaminate the body-wearable medical device itself. When contaminating the body-wearable medical device, in particular when covering sensor elements or electronics or when entering the body-wearable medical device the body fluid may cause a malfunction of the body-wearable medical device and may also pose a hygienic risk. Furthermore, body fluid flowing out of the insertion lesion may reduce the adhesion of the adhesive of the first side of the base portion of the body-wearable medical device. Thus, there is a chance that the body-wearable medical device could unintentionally detach from the patient's body.

### SUMMARY

This disclosure teaches a body-wearable medical device that avoids at least one of the problems associated with medical devices known from prior art. In particular, this disclosure teaches a body-wearable medical device that is less prone to malfunction caused by contamination by body fluid flowing out of the insertion lesion and that is less prone to unintentionally detach from a patient's body.

According to a first aspect, a body-wearable medical device is provided. The body-wearable medical device comprises a medical device housing comprising a transcutaneous element that is configured for being at least partially inserted into a patient's body at an insertion site during use of the body-wearable medical device, and a base portion that is configured for reversibly attaching the body-wearable medical device during its use to a patient's skin. The base portion comprises a first side that during use of the body-wearable medical device faces the patient's body and that comprises an adhesive for adhering the body-wearable medical device to a patient's skin. The base portion on its first side further comprises a superabsorbent substance and at least one fluid channel, the at least one fluid channel being configured to guide body fluid from the insertion site to the superabsorbent substance.

When being reversibly attached to a patient's skin by means of the adhesive of the first side of the base portion, the first side of the base portion faces the patient's body. A non-exhaustive list of examples of adhesives include, but are not limited to, acrylic-based, dextrin-based, urethane-based, rubber-based, polyvinyl-ether-based adhesives, and other adhesives based on natural and synthetic elastomers.

A second side of the base portion is arranged opposite to the first side, i.e., forming a side of the base portion that during use of the body-wearable medical device faces away from the patient's skin. The second side is attached to the medical device housing, such that the medical device housing during use of the body-wearable medical device is reversibly attached to a patient's skin via the base portion. For example, the medical device housing is attached to the second side by an adhesive or by ultrasound welding.

By being comprised on the first side of the base portion, the superabsorbent substance as well as the at least one fluid channel, thus, also faces the patient's body, such that a body fluid which may flow out of the insertion lesion caused by the transcutaneous element is guided by the at least one fluid channel to the superabsorbent substance. By guiding the body fluids from the insertion site to the superabsorbent substance, the fluid channel facilitates the superabsorbent substance absorbing the body fluids and helps prevent the body fluids from bypassing the superabsorbent substance. According to an embodiment, the fluid channel is formed by silicone, directionally aligned fibers, directionally aligned capillary tubes, cellulose, or foam or a combination of the aforementioned. Without being bound to a particular theory, in an embodiment of this disclosure, capillary forces guide body fluid from the insertion site to the superabsorbent substance. By way of example, the at least one fluid channel can be formed by a silicone structure that is configured to guide body fluids from the insertion site to the superabsorbent substance. The silicone structure can at least partially projects from and/or at least being integrated into the first side of the base portion, such that during use of the body-wearable medical device, the silicone structure is in contact with the skin of the user and the base portion. The silicone structure together with the base portion and the skin form a tunnel-shaped channel that guides a flow of body fluid from the insertion site to the superabsorbent substance.

After being guided to the superabsorbent substance, the body fluid is at least partially absorbed by the superabsorbent substance. Accordingly, the superabsorbent substance is configured for absorbing body fluids flowing out of an insertion lesion caused by the transcutaneous element. This is associated with the advantage that the body fluid absorbed by the superabsorbent substance is retained therein and, thus, cannot contaminate the body-wearable medical device and/or the patient's clothes. Furthermore, the absorbed and retained body fluid does not adversely affect the adhesion of the adhesive and thus prevents the body-wearable medical device from being unintentionally detached from a patient's body.

In the context of this disclosure, a superabsorbent substance is able to absorb and retain large volumes of water in aqueous solutions. A superabsorbent substance according to this disclosure is, for example, modified starch, polymerized polyvinyl alcohol (PVA) and polyethylene oxide (PEO) that are hydrophilic and have a high affinity to water. When being chemically or physically cross-linked, these superabsorbent substances become water-swellable but not water-soluble. In an embodiment, the superabsorbent substance is a superabsorbent polymer in the form of granular particles, fibers, or a mixture thereof. In a further variant, the superabsorbent polymer is made by polymerization of acrylic acids blended with sodium hydroxide in the presence of an initiated poly-acrylic acid sodium salt. Alternatively, the superabsorbent polymer can be an ethylene maleic anhydride copolymer, a cross-linked carboxymethylcellulose, a polyvinyl alcohol copolymer, a cross-linked polyethylene oxide, and a starch-grafted copolymer of polyacrylonitrile. Superabsorbent polymers can be made by standard methods such as by gel polymerization, suspension polymerization or solution polymerization.

According to an embodiment, a body-wearable medical device is one of a drug delivery device such as an insulin pump, or a medical device that is configured for analyzing body fluid of a patient, in particular medical device for measuring an analyte such as a continuous glucose-monitoring device.

In an embodiment, the transcutaneous element is a cannula, for example, a soft cannula or a steel cannula, a probe that is configured for retrieving body fluid, or a sensor that is configured for analyzing body fluid. Presence of a transcutaneous element does not exclude a presence of additional elements. For example, in a case in which the transcutaneous element is a cannula of a drug delivery device or of an insulin pump, respectively, the drug delivery device or the insulin pump may further comprise an additional transcutaneous element such as, for example, a probe or sensor of the type as outlined above. Likewise, in the case of a continuous glucose-monitoring device, the transcutaneous element is a probe or a sensor of the type as outlined above, and the continuous glucose monitoring device may further comprise an additional probe or a sensor or a cannula of a type as outlined above.

According to an embodiment, the body-wearable medical device is a drug delivery device or an insulin pump, wherein the transcutaneous element is a cannula, for example, a soft cannula or a steel cannula. In another embodiment, the body-wearable medical device is a continuous glucose monitoring device, wherein the transcutaneous element is a probe or sensor of a type as outlined above, i.e., a probe that is configured for retrieving body fluid, or a sensor that is configured for analyzing body fluid.

Apart from comprising an adhesive, in an embodiment, the base portion further comprises a pad that during use of the body-wearable medical device is adhered to the patient's body by means of the adhesive. In order to improve the handling of the body-wearable medical device and to prevent the adhesive from being unintentionally worn, the adhesive can be covered by one or more backing layers that have to be peeled off from the adhesive before use of the body-wearable medical device. The pad can comprise one or more of a sterile non-woven, cellulose, tissue or paper.

Additionally or alternatively, in an embodiment, the base portion is vapor permeable and/or comprises a vapor permeable membrane that is configured to allow vapor to flow through the base portion and/or the vapor permeable membrane. Moisture that, during use of the body-wearable medical device, may be prone to accumulate in or underneath the base portion can at least partially evaporate through the base portion and/or the vapor permeable membrane. Evaporation of vapor through the base portion and/or the vapor permeable membrane helps provide a wound-healing and/or skin-friendly environment around the insertion site and underneath the body-wearable medical device. Furthermore, evaporation of moisture helps reduce accumulation of moisture underneath the body-wearable medical device, and, thus, helps hinder unintended detachment of the body-wearable medical device.

In an embodiment, the superabsorbent substance at least partially circumscribes the transcutaneous element. In the context of this disclosure, at least partially circumscribing means that the superabsorbent substance comprises segments that are arranged on the first side of the base portion in at least two different angular positions relative to the transcutaneous element. The superabsorbent substance can form a continuous structure or a discontinuous structure. In the context of this disclosure, a continuous structure is a structure that circumscribes the transcutaneous element without having a gap therein through which body fluid flowing out of the insertion site along the first side of the base portion can pass the superabsorbent structure without moisten the superabsorbent substance.

In an embodiment, the superabsorbent substance forms an enclosure on the first side of the base portion, wherein the superabsorbent substance circumscribes the transcutaneous element. An enclosure that circumscribes the transcutaneous element is a continuous structure surrounding the transcutaneous element. The superabsorbent substance is configured for absorbing and retaining body fluids, and may also form a barrier preventing body fluids from flowing beyond the area that is enclosed by the superabsorbent substance. According to an embodiment, the enclosure forms a ring that circumscribes the transcutaneous element.

According to an embodiment of the body-wearable medical device, the superabsorbent substance on the first side of the base portion forms a discontinuous enclosure by having multiple enclosure segments that are displaced from each other along a first virtual enclosure around the medical device housing. The enclosure is discontinuously formed on the first side of the base portion by multiple enclosure segments that are displaced from each along a first virtual enclosure. The first virtual enclosure circumscribes the transcutaneous element along a geometric shape. A non-exhaustive group of geometric shapes comprises by way of example a ring, an ellipse, a rectangle and a polygon. By being displaced from each other along the first virtual enclosure, there is a gap between neighboring enclosure segments. The spacing between neighboring enclosure segments helps the structure formed by the superabsorbent substance to be more flexibly adapted to the body shape at the insertion site. In an embodiment, the multiple enclosure segments are ring segments that are displaced from each other along a first virtual ring. In an embodiment, each segment is connected to at least one of the at least one fluid channel, in another embodiment each segment is connected to 1 to 4 fluid channels, or to 1 to 3 fluid channels or to 1 to 2 fluid channels or to 1 fluid channel.

In an embodiment, the superabsorbent substance on the first side of the base portion forms a continuous or discontinuous inner enclosure and a continuous or discontinuous outer enclosure. The inner and outer enclosure, both form a barrier for absorbing and retaining body fluids flowing out of an insertion lesion during use of the body-wearable medical device. Since the inner and outer enclosures at least partially circumscribe the transcutaneous element, the terms “inner” and “outer” refer to the radial distance of the respective inner enclosure and outer enclosure from the transcutaneous element along the first side of the barrier. According to an embodiment, the inner enclosure is continuously formed along a first virtual enclosure, while the outer enclosure is discontinuously formed along a second virtual enclosure. In an embodiment, the distance between the insertion site and the most distant part of the inner enclosure is smaller than the distance between the insertion side and the closest distant part of the outer enclosure. In an embodiment the inner and outer enclosure are concentrically arranged with transcutaneous element in the center. The inner enclosure and the outer enclosure can form two concentric rings. In an embodiment, the inner enclosure is discontinuously formed along a first virtual enclosure, while the outer enclosure is continuously formed along a second virtual enclosure. In yet another embodiment, the inner enclosure is continuously formed along a first virtual enclosure and the outer enclosure is continuously formed along a second virtual enclosure. In another embodiment, the inner enclosure is discontinuously formed along a first virtual enclosure and the outer enclosure is continuously formed along a second virtual enclosure.

In an embodiment, the superabsorbent substance on the first side of the base portion forms a discontinuous inner enclosure having multiple inner enclosure segments and a discontinuous outer enclosure having multiple outer enclosure segments, wherein the multiple inner enclosure segments and the multiple outer enclosure segments are alternately arranged with respect to an angular position relative to the transcutaneous element along a first virtual enclosure and along a second virtual enclosure. The inner enclosure and the outer enclosure together form a barrier for absorbing and retaining body fluids originating from an insertion lesion caused by the transcutaneous element. The multiple inner enclosure segments and the multiple outer enclosure segments are arranged on the first side of the base portion along a first virtual enclosure and a second virtual enclosure, respectively, wherein the inner enclosure segments and the outer enclosure segments are alternately arranged at different angular positions relative to the transcutaneous element. In an embodiment, the inner enclosure segments and the outer enclosure segments are formed by ring segments.

According to an embodiment, the base portion further comprises a septum that is configured for being pierced by the transcutaneous element during use of the body-wearable medical device. During use of the body-wearable medical device, the septum of the base portion is traversed by the transcutaneous element. According to an embodiment, the septum is a self-sealing septum i.e., when the transcutaneous element (such as a cannula, sensor, etc.) has been inserted through the septum and is afterwards removed, the septum will close and forms a barrier for microorganisms and body fluids. The self-scaling septum can, for example, be made of silicone, plastic, polyurethane foam or combination thereof.

In an embodiment, the first side of the base portion further comprises an outer sealing element that circumscribes the superabsorbent substance on a side of the superabsorbent substance facing away from the transcutaneous element. The outer scaling element helps to retain body fluid within the area encircled by the outer sealing element. The outer sealing elements form a barrier in addition to the superabsorbent substance that forms a first barrier for body fluids. In contrast to the superabsorbent substance, the outer sealing element in the context of this disclosure does not significantly absorb body fluid when compared with the superabsorbent substance. In an embodiment, the outer scaling element is formed by a hydrophobic material. By being arranged on an outer side of the superabsorbent substance, i.e., the side of the superabsorbent substance facing away from the transcutaneous element, the sealing element forms an outer boundary, when being viewed from the perspective of the transcutaneous element.

According to an embodiment, the first side of the base portion further comprises an inner sealing element on a side of the superabsorbent substance facing the transcutaneous element. The sealing element forms an inner barrier helping to retain the body fluid within the area encircled by the inner sealing element. The inner sealing element being arranged on a side of the superabsorbent substance facing the transcutaneous element forms an inner barrier. The inner sealing element encircles an area of the first side of the base portion containing the transcutaneous element. The superabsorbent substance is arranged outside of the area encircled by the sealing that is arranged on a side of the superabsorbent substance facing the transcutaneous element.

In an embodiment, the inner sealing element and/or the outer sealing element, if present, is formed by or comprises a silicone structure that at least partially projects away from the first side of the base portion and/or is integrated in the first side of the base portion, such that, during use of the body-wearable medical device, the silicone structure is in contact with the base portion and the skin of the user. By way of example, the inner sealing element and/or the outer sealing element can be made of or comprise a lip or a structure that, during use of the body-wearable medical device, at least partially projects from the first side of the base portion towards the skin of a user or that is in contact with the skin of a user. The lip or structure can be, by way of example, a silicone lip or a silicone structure that forms a physical barrier that hinders body fluid passing the barrier.

In an embodiment, the first side of the base portion comprises a plaster that carries the adhesive for attaching the body-wearable medical device to a patient's body. The plaster can help to adhere the body-wearable medical device to the body shape at the insertion side.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows a cross-section of a body-wearable medical device;

FIG. 2 shows a top view of a body-wearable medical device;

FIG. 3 shows a top view of a body-wearable medical device;

FIG. 4 shows a cross-section of a body-wearable medical device;

FIG. 5 shows a cross-section of a body-wearable medical device;

FIG. 6 shows a cross-section of a body-wearable medical device; and

FIG. 7 shows a top view of a body-wearable medical device.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

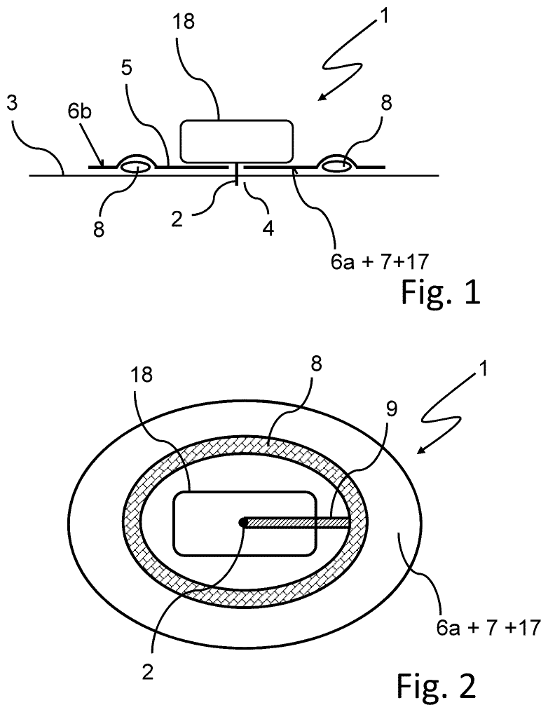

FIGS. 1 and 2 show a first embodiment, wherein FIG. 1 shows a cross-section of the body-wearable medical device 1 while FIG. 2 shows a top view of the body-wearable medical device 1. The body-wearable medical device 1 comprises a medical device housing 18 and a transcutaneous element 2. The transcutaneous element 2 is configured for being at least partially inserted into the skin of a patient's body 3 at an insertion site 4 during use of the body-wearable medical device 1. Here the transcutaneous element 2 is a cannula by way of example. Alternatively, the transcutaneous element 2 can be a probe that is configured for retrieving body fluid or a sensor that is configured for analyzing body fluid.

In FIG. 1, the patient's body 3 and the insertion site 4 are shown to explain the intended use of the body-wearable medical device 1 by way of an example.

The body-wearable medical device 1 further comprises a base portion 5. The base portion 5 has a first side 6a that during use of the body-wearable medical device 1 faces the patient's body 3. For adhering the body-wearable medical device 1 during its use to a patient's skin, the first side 6a of the base portion 5 further comprises an adhesive 7. The base portion 5 further comprises a second side 6b opposite to the first side 6a and, during use, facing away from the patient's body. The second side 6b is coupled to the medical device housing 18.

When being at least partially inserted into the patient's body 3, the transcutaneous element 2 forms an opening in the patient's skin. Body fluids flowing out of this opening may contaminate or negatively affect the functionality of the body-wearable medical device 1 or may contaminate the patient's clothes. However, for absorbing and retaining body fluids that, during use of the body-wearable medical device 1 may flow out of an opening caused by the transcutaneous element 2, a superabsorbent substance 8 and fluid channel 9 is provided on the first side 6a of the base portion 5 of the body-wearable medical device 1. The fluid channel 9 guides the body fluids from the insertion site 4 towards the superabsorbent substance 8.

As shown in the top view of FIG. 2, the superabsorbent substance by way of example may form a continuous structure circumscribing the transcutaneous element 2 of the body-wearable medical device 1. The adhesive 7 for adhering the body-wearable medical device 1 to a patient's skin is carried by a plaster 17 that is provided on the first side 6a of the body-wearable medical device 1. In the top view, the plaster 17 extends beyond the projection of the base portion 5 of the body-wearable medical device 1.

FIG. 3 shows a top view of a body-wearable medical device 1 according to another embodiment. The embodiment of FIG. 3 differs from the embodiment of the body-wearable medical device 1 as shown in FIGS. 1 and 2 by additionally comprising a fluid channel 9. Fluid channel 9 is configured for guiding body fluids from the insertion site 4 to the superabsorbent substance 8. Here the fluid channel 9 comprise four arms forming a cross and which funnel body fluid from the insertion site 4 of the transcutaneous element 2 into the superabsorbent substance 8.

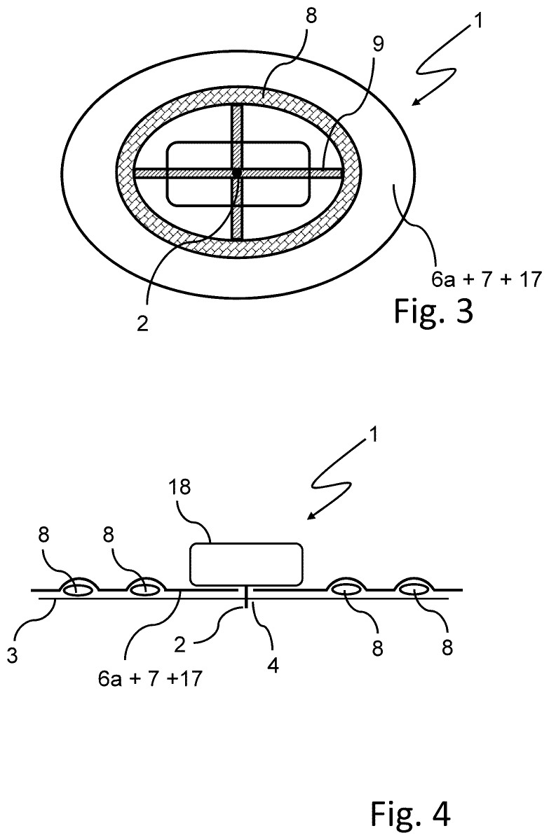

FIG. 4 shows a cross-section of a body-wearable medical device 1 that differs from the embodiment according to FIGS. 1 and 2 in having a differently shaped superabsorbent substance 8. Here, the superabsorbent substance 8 forms a continuous inner enclosure and a continuous outer enclosure, both circumscribing the transcutaneous element 2 along the first side 6a of the base portion 5. The continuous inner enclosure and the continuous outer enclosure form two concentrically circles, wherein the common center of the two circles is formed by the transcutaneous element 2.

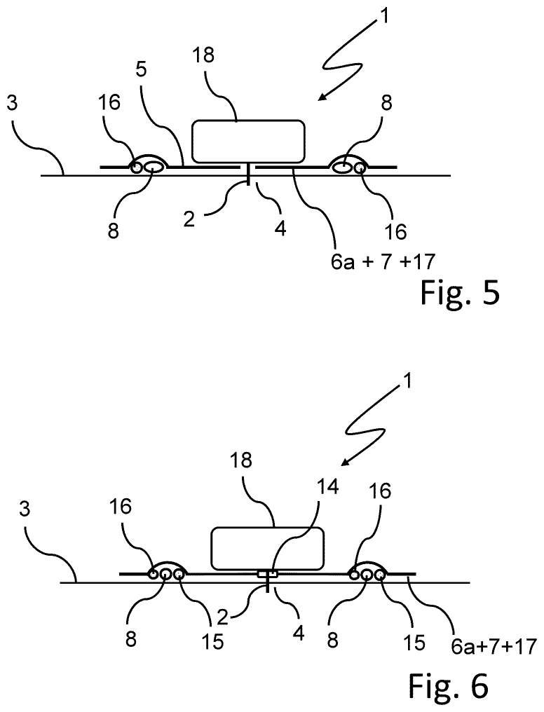

FIG. 5 shows a cross-section of a further embodiment of the body-wearable medical device 1. The body-wearable medical device 1 according to the embodiment of FIG. 5 differs from the embodiment according to FIG. 1 by having an outer sealing element 16. The outer sealing element 16 circumscribes the superabsorbent substance 8 on a side of the superabsorbent substance 8 facing away from the transcutaneous element 2. Accordingly, the superabsorbent substance 8 as well as the transcutaneous element 2 is encircled by the outer sealing element 16, and thus arranged within an area of the first side 6a of the base portion 5 circumscribed by the outer sealing element 16.

FIG. 6 shows a cross-section of yet another embodiment of the body-wearable medical device 1. The body-wearable medical device 1 of FIG. 6 differs from the body-wearable medical device 1 of FIG. 5 by additionally having an inner sealing element 15. The inner sealing element 15 circumscribes the transcutaneous element 2 on a side of the superabsorbent substance 8 that faces the transcutaneous element 2, and thus is placed in a smaller radial distance from the transcutaneous element 2 than the superabsorbent substance 8. The first side 1 of the base portion 5 of the body-wearable medical device 1 according to FIG. 6 further additionally comprises a septum 14. Septum 14 is configured for being pierced by the transcutaneous element 2 during use of the body-wearable medical device 1.

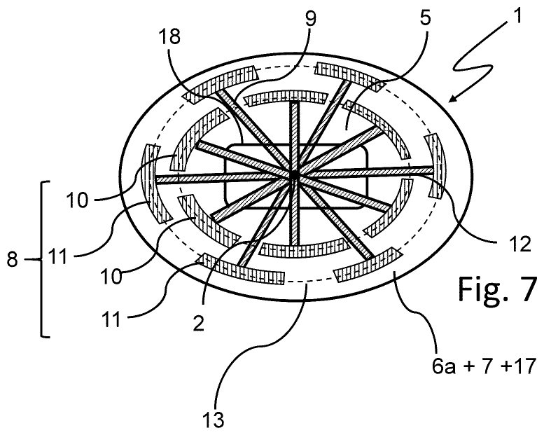

FIG. 7 shows a top view of a body-wearable medical device 1 according to another embodiment. The body-wearable medical device 1 of FIG. 7 differs from the body-wearable medical device 1 according to FIG. 1 by having a superabsorbent substance 8 that forms a discontinuous inner enclosure and a discontinuous outer enclosure. The discontinuous inner enclosure is formed by multiple inner enclosure segments 10, and the discontinuous outer enclosure 12 are formed by multiple outer enclosure segments 11. The multiple inner enclosure segments 10 are arranged on the first side 6a of the base portion 5 along a first virtual enclosure 12, while the multiple outer enclosure segments 11 are arranged on the first side 6a of the base portion 5 along a second virtual enclosure 13. The inner enclosure segments 10 and the outer enclosure segments 11 are alternately arranged at different angular positions with respect to the transcutaneous element 2. Each of the inner enclosure segments 10 and each of the outer enclosure segments 11 are connected to a fluid channel 9, wherein each of the fluid channels 9 is configured to guide body fluid flowing out of the insertion site to the superabsorbent substance 8 of the respective segments 10, 11.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

1 Body-wearable medical device
2 Transcutaneous element
3 Patient's body
4 Insertion site
5 Base portion
6a First side of base portion
6b Second side of base portion
7 Adhesive
8 Superabsorbent substance
9 Fluid channel
10 Inner enclosure segments
11 Outer enclosure segments
12 First virtual enclosure
13 Second virtual enclosure
14 Septum
15 Inner sealing element
16 Outer sealing element
17 Plaster
18 Medical device housing

What is claimed is:

1. A body-wearable medical device, comprising:
a housing having a transcutaneous element configured to be at least partially inserted into a patient's body at an insertion site during use of the body-wearable medical device; and
a base configured for reversibly attaching the body-wearable medical device housing to a patient's skin;
wherein the base has a first side that during use of the body-wearable medical device faces the patient's body and that also has an adhesive for configured to adhere the body-wearable medical device housing to the patient's skin;
wherein the base on its first side has a superabsorbent substance and at least one fluid channel configured to guide body fluid from the insertion site to the superabsorbent substance.

2. The body-wearable medical device according to claim 1, wherein the superabsorbent substance at least partially circumscribes the transcutaneous element.

3. The body-wearable medical device according to claim 1, wherein the superabsorbent substance forms an enclosure on the first side of the base portion and circumscribes the transcutaneous element.

4. The body-wearable medical device according to claim 1, wherein the superabsorbent substance on the first side of the base portion forms a discontinuous enclosure formed by multiple enclosure segments that are spaced from each other along a first virtual enclosure.

5. The body-wearable medical device according to claim 1, wherein the superabsorbent substance on the first side of the base forms a continuous or discontinuous inner enclosure and a continuous or discontinuous outer enclosure.

6. The body-wearable medical device according to claim 1, wherein the superabsorbent substance on the first side of the base forms a discontinuous inner enclosure having multiple inner enclosure segments and a discontinuous outer enclosure having multiple outer enclosure segments, wherein the multiple inner enclosure segments and the multiple outer enclosure segments are alternately arranged with respect to an angular position relative to the transcutaneous element along a first virtual enclosure and along a second virtual enclosure.

7. The body-wearable medical device according to claim 1, wherein the base further comprises a septum that is configured for being pierced by the transcutaneous element during use of the body-wearable medical device.

8. The body-wearable medical device according to claim 1, wherein the first side of the base further comprises an outer seal that circumscribes the superabsorbent substance on a side of the superabsorbent substance facing away from the transcutaneous element.

9. The body-wearable medical device according to claim 8, wherein the outer seal comprises a silicone structure that at least partially projects away from the first side of the base portion and/or is integrated in the first side of the base, such that, during use of the body-wearable medical device, the silicone structure is in contact with the base and the skin of the user.

10. The body-wearable medical device according to claim 1, wherein the first side of the base further comprises an inner seal on a side of the superabsorbent substance facing the transcutaneous element.

11. The body-wearable medical device according to claim 10, wherein the inner seal comprises a silicone structure that at least partially projects away from the first side of the base and/or is integrated in the first side of the base, such that, during use of the body-wearable medical device, the silicone structure is in contact with the base and the skin of the user.

12. The body-wearable medical device according to claim 1, wherein the first side of the base comprises plaster that carries the adhesive for attaching the body-wearable medical device to a patient's body.

13. The body-wearable medical device according to claim 1, wherein each of the inner enclosure segments and outer enclosure segments is connected to at least one of the at least one fluid channel.
